(19) United States
(12) Patent Application Publication
(10) Pub. No.: US 2024/0174713 A1
(43) Pub. Date: May 30, 2024

(54) PEPTIDE SYNTHESIS METHOD INVOLVING STERICALLY HINDERED MIXED ANHYDRIDE INTERMEDIATE

(71) Applicant: AMICOAT AS, Tromsø (NO)

(72) Inventors: Jacobus Johannes Eksteen, Tromso (NO); John Sigurd Svendsen, Sykehusvegen (NO); Sophie Borghese, Bussels (BE); Florence Malmedy, Brussels (BE); Martin Bousmanne, Woluwe Saint-Lambert (BE)

(21) Appl. No.: 18/575,343

(22) PCT Filed: Jul. 4, 2022
(86) PCT No.: PCT/EP2022/068484

§ 371 Date: Dec. 29, 2023

(30) Foreign Application Priority Data
Jul. 2, 2021 (GB) 2109626.8

(51) Int. Cl. C07K 5/09; C07K 1/00

(52) U.S. Cl. CPC C07K 5/0817; C07K 1/003

(57) ABSTRACT

The invention is directed to a method of making a target peptide comprising reacting a mixed anhydride compound of Formula (I) with a second moiety which is an amino acid or peptide, wherein Formula (I) has the following structure:

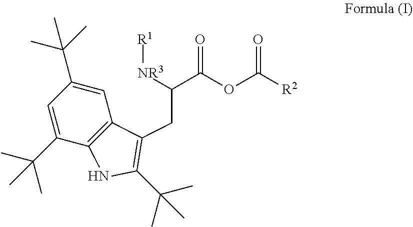

and wherein R¹-R³ are as defined in the disclosure.

### TECHNICAL FIELD

The invention is directed to a method of making a target peptide.

### BACKGROUND

At laboratory scale, many coupling strategies are available for the production of peptides. However, most of these are too expensive for commercial scale production. Coupling reactions between amino acids are, almost exclusively, facilitated by activation of the carboxylic acid of the incoming amino acid. For example, O-(1H-Benzotriazol-1-yl)-N,N,N′,N′-tetramethyluronium hexafluorophosphate (HTBU) is an efficient activator that gives minimal racemization. However, the cost of HTBU is high.

It would be desirable to be able to use lower cost activators without compromising on selectivity or reaction rate. However, lower cost activators are not suitable for all peptide coupling strategies. In particular, depending on the candidate amino acid to be activated, some activators may not allow coupling at acceptable rates or with acceptable selectivity. Slow reactions in particular may be associated with undesirable racemization/epimerisation.

As illustrated in FIG. 22 of Rehman et al., Side reactions in peptide synthesis: An overview, International Journal of Pharmaceutical Research & Technology, Volume—10, 2018, when a candidate amino acid contains a sterically bulky side chain, activators that form mixed anhydride intermediates do not usually provide acceptable selectivity, since the amino nucleophile has a better chance to attack on the undesired carbonyl group which does not result in amide bond formation.

### SUMMARY OF THE INVENTION

The present inventors have surprisingly found that mixed anhydride peptide synthesis can be used and will provide yields suitable for a commercial process even when the peptides incorporate an amino acid with a sterically bulky side chain. Thus, in one aspect, the invention provides a method for making a target peptide comprising reacting a mixed anhydride compound of Formula (I) with a second moiety which is an amino acid or peptide;
wherein Formula (I) has the structure:

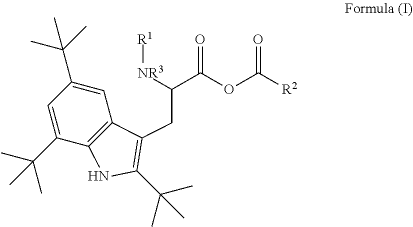

wherein R¹ is a protecting group, a peptide or an amino acid;
wherein R² is tert-butyl, isobutoxy, tert-butoxy, isobutyl, isopropoxy, isopropyl, or ethoxy; and
wherein R³ is H or an alkylsilyl group.

In another aspect, the invention provides a method of peptide synthesis comprising reacting a mixed anhydride compound of Formula (I) with a second moiety which is an amino acid or peptide. The method may be a step in the synthesis of a target peptide. Embodiments of other aspects of the invention described herein apply, mutatis mutandis, to this aspect of the invention.

In another aspect, the invention is directed to a compound of Formula (I). Embodiments of other aspects of the invention described herein apply, mutatis mutandis, to this aspect of the invention.

### BRIEF DESCRIPTION OF THE FIGS.

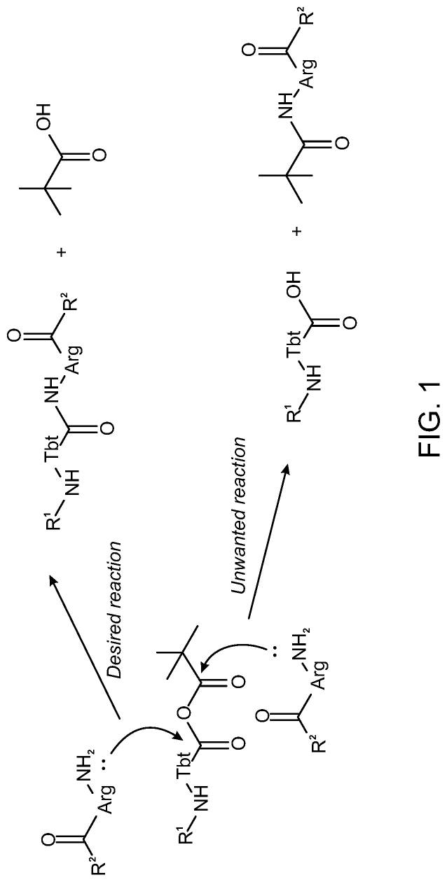

FIG. 1 shows two possible reactions between an exemplary mixed anhydride compound and an amino group of an exemplary second moiety. The desired reaction (upper arrow) involves the carbonyl closest to the tri-tert-butyl-tryptophan (Tbt) side chain. The undesired reaction (bottom arrow) involves reaction at the “wrong” carbonyl group of the mixed anhydride compound and does not result in the formation of the desired amide bond. The second moiety in FIG. 1 comprises an arginine residue and R² in FIG. 1 represents the rest of the second moiety.

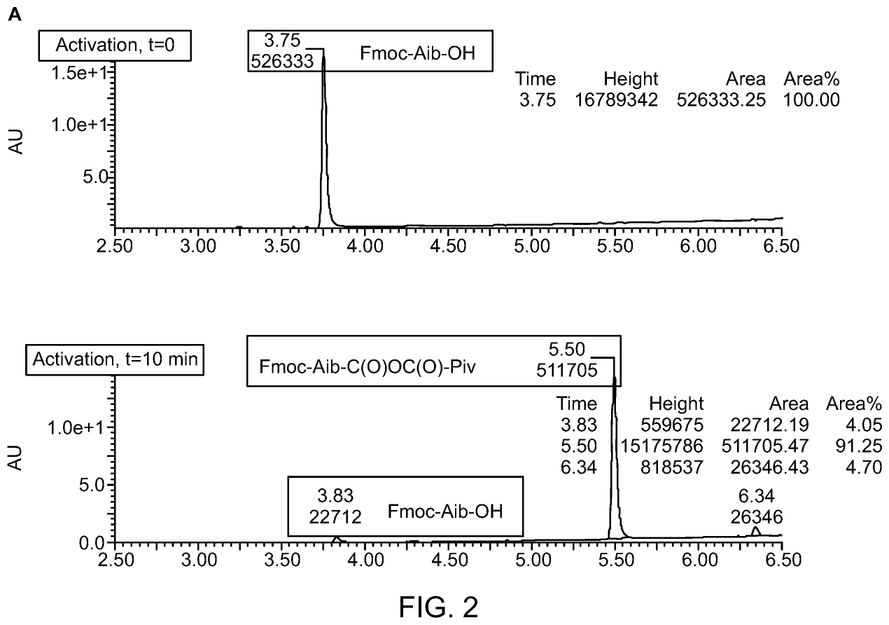

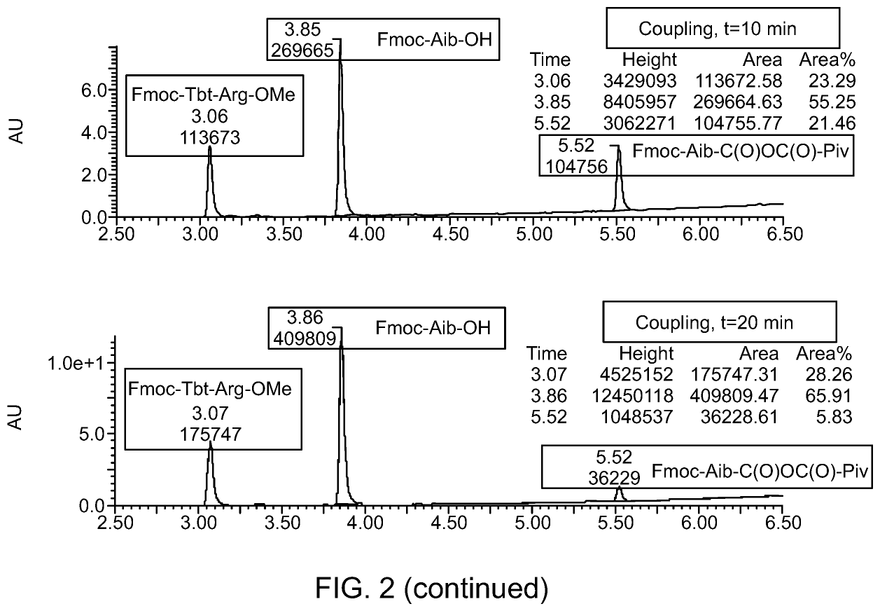

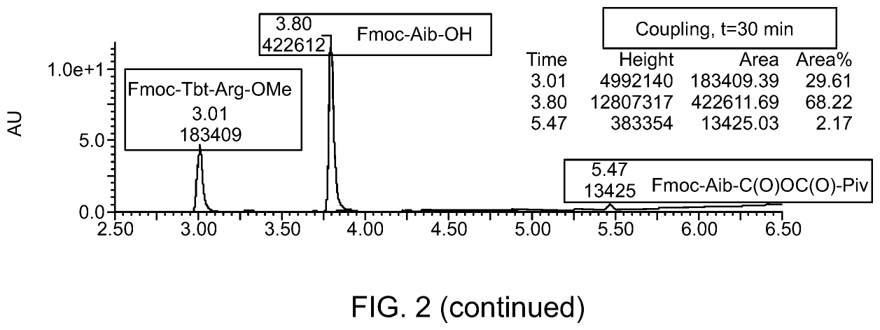

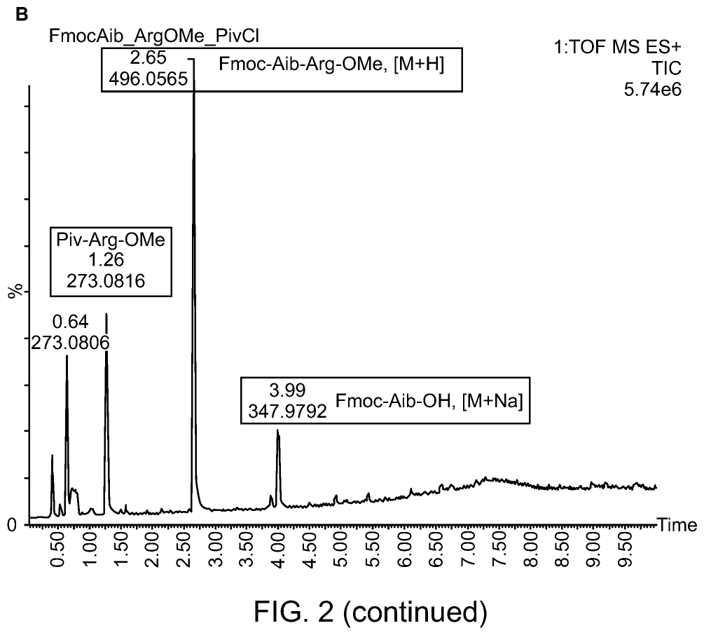

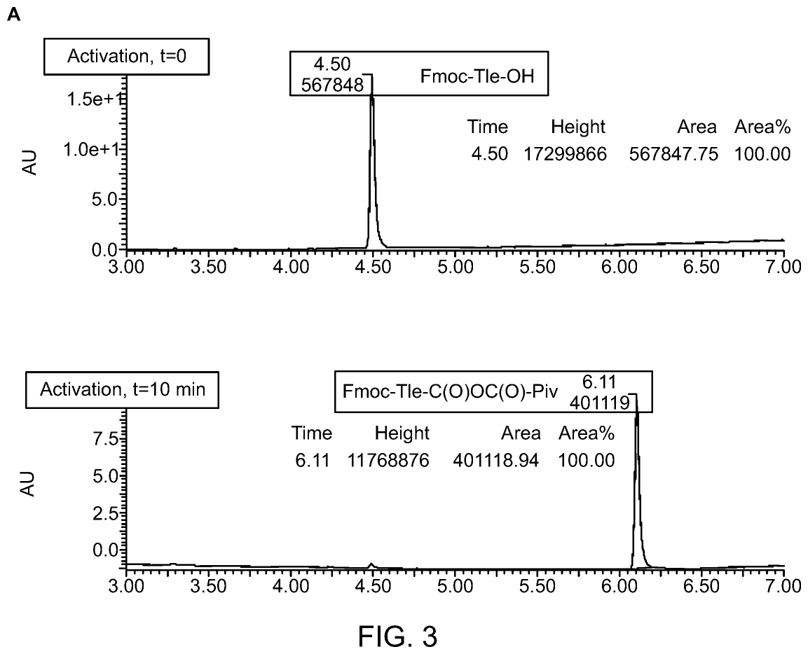

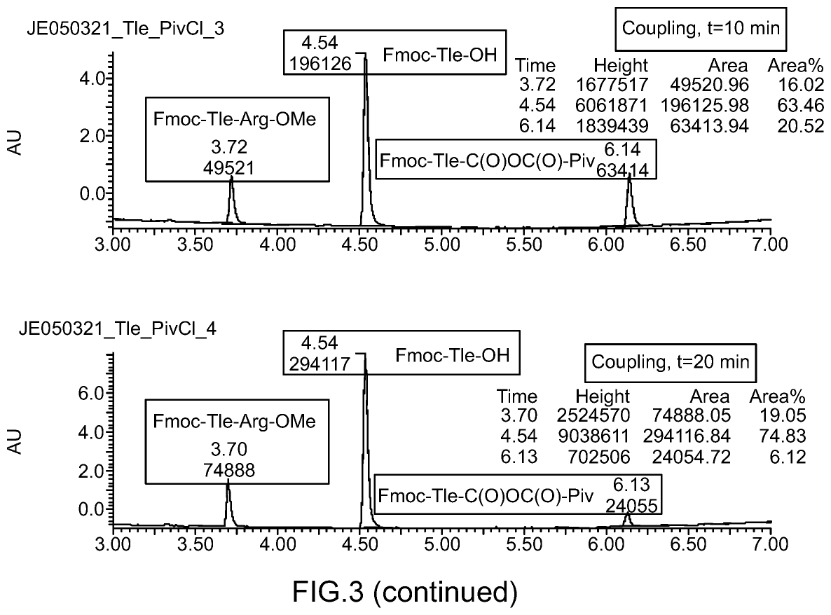

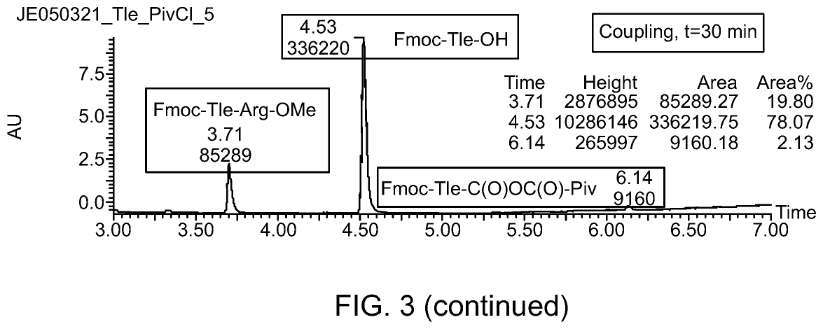

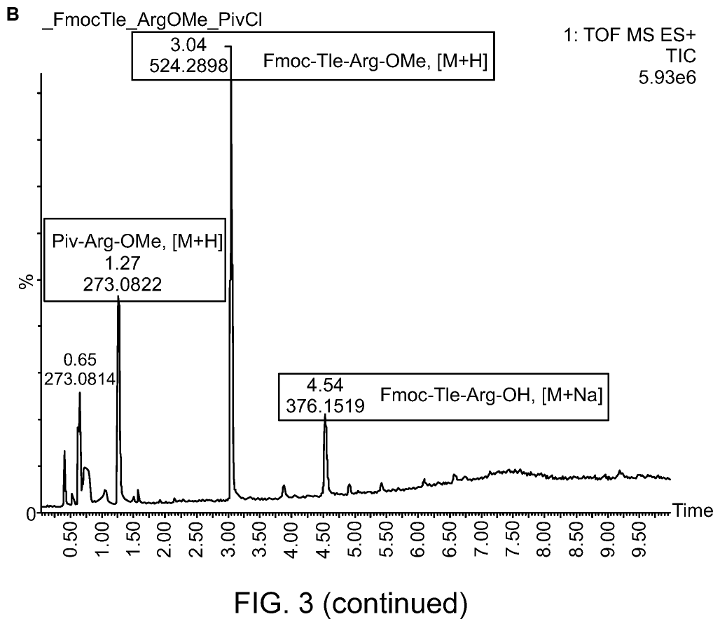

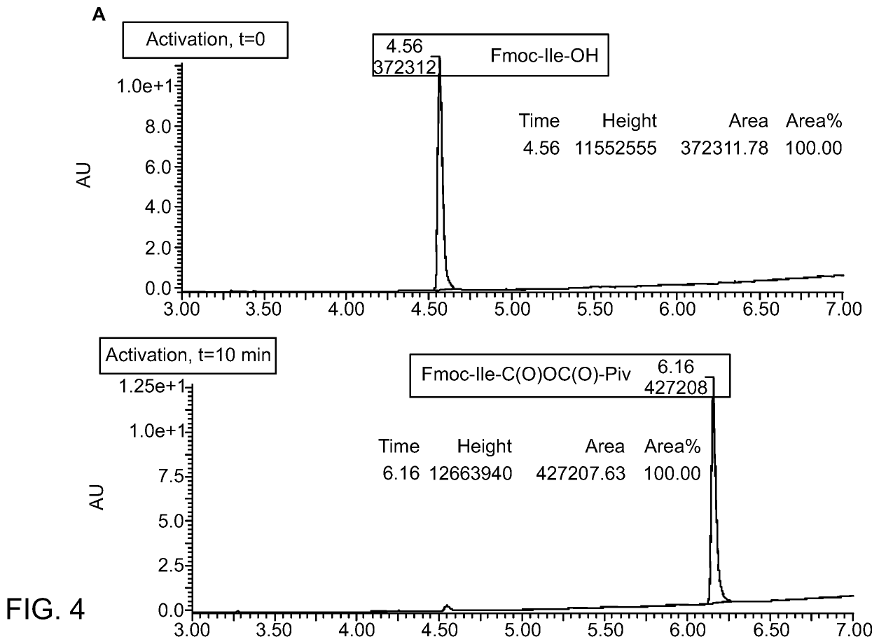

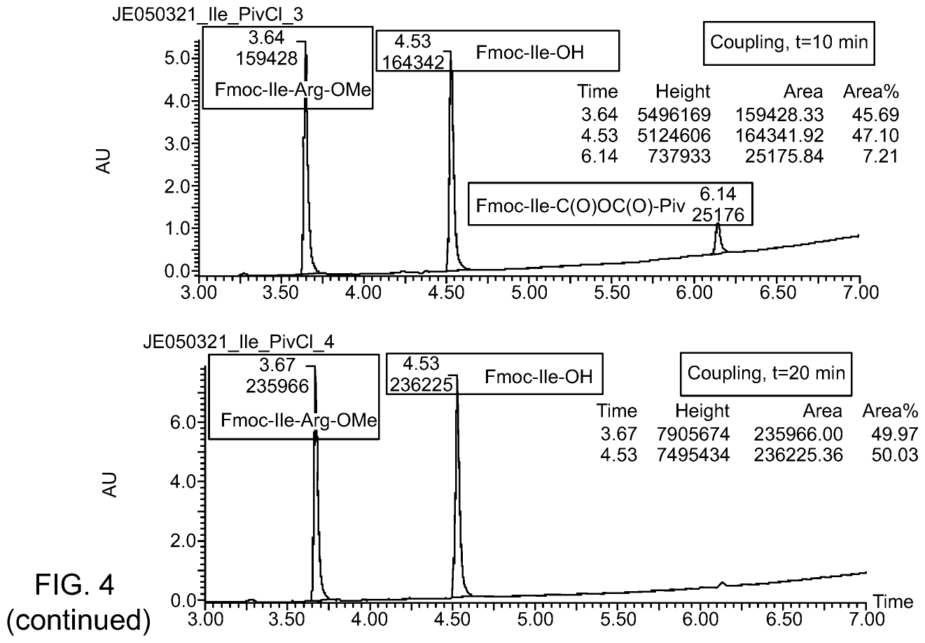

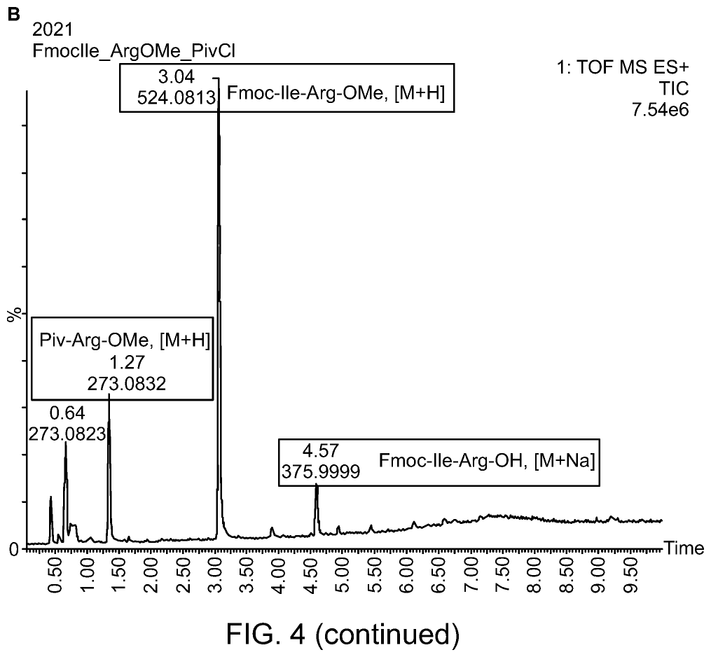

FIGS. 2A-4A show UPLC-PDA traces of the activation and coupling of Fmoc-Aib-OH (FIG. 2A), Fmoc-Tle-OH (FIG. 3A), and Fmoc-Ile-OH (FIG. 4A) to H-Arg-OMe using pivaloyl chloride.

FIGS. 2B-4B show UPLC-MS spectra after completion of the coupling reaction of Fmoc-Aib-OH (FIG. 2B), Fmoc-Tle-OH (FIG. 3B), and Fmoc-Ile-OH (FIG. 4B) to H-Arg-OMe using pivaloyl chloride.

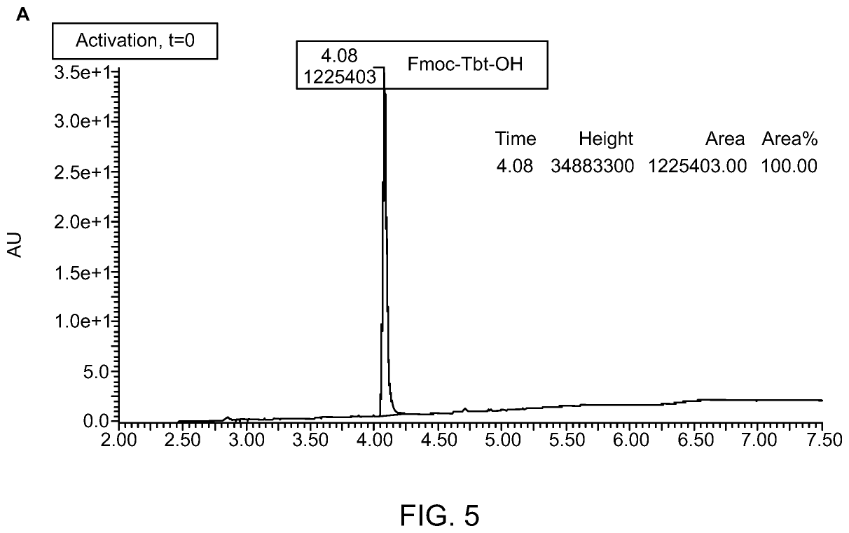

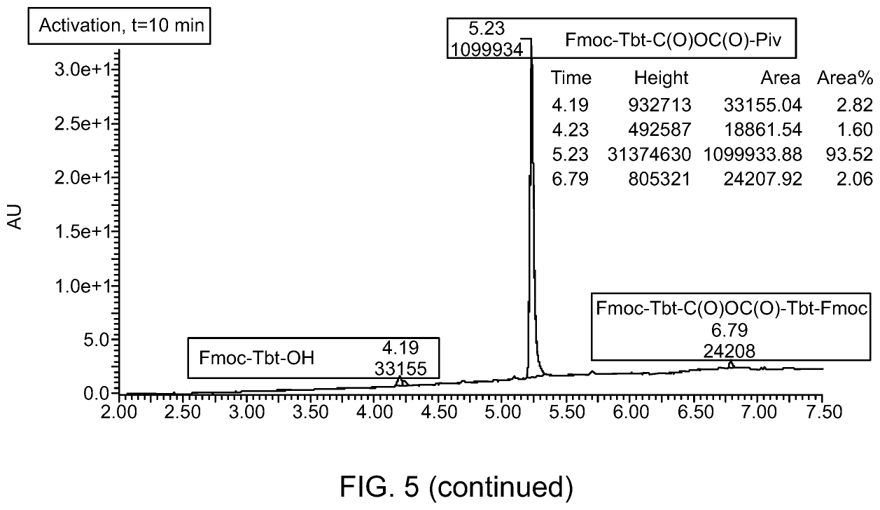

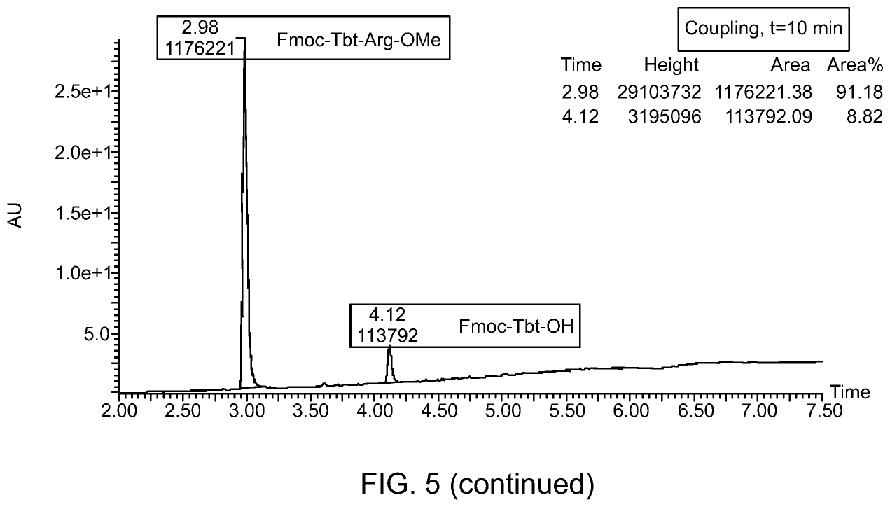

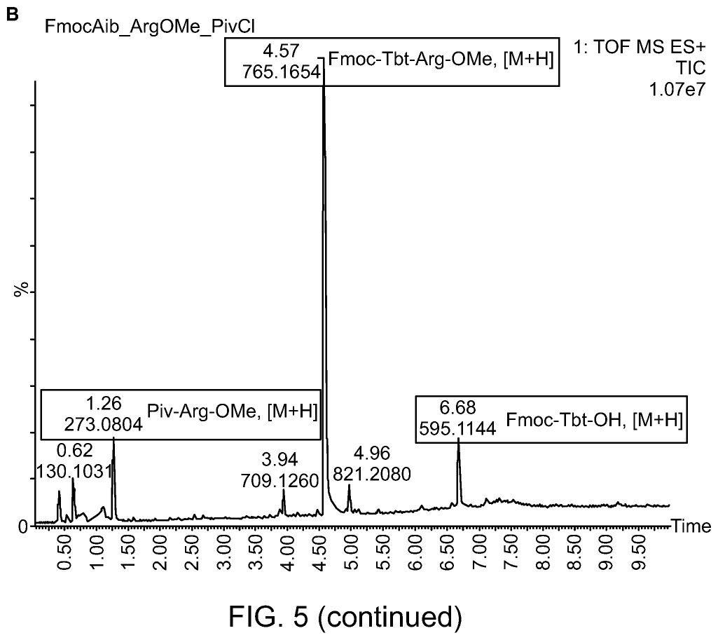

FIG. 5 shows UPLC-PDA traces (FIG. 5A) of the activation and coupling of Fmoc-Tbt-OH to H-Arg-OMe using pivaloyl chloride and UPLC-MS spectra (FIG. 5B) after completion of the coupling reaction.

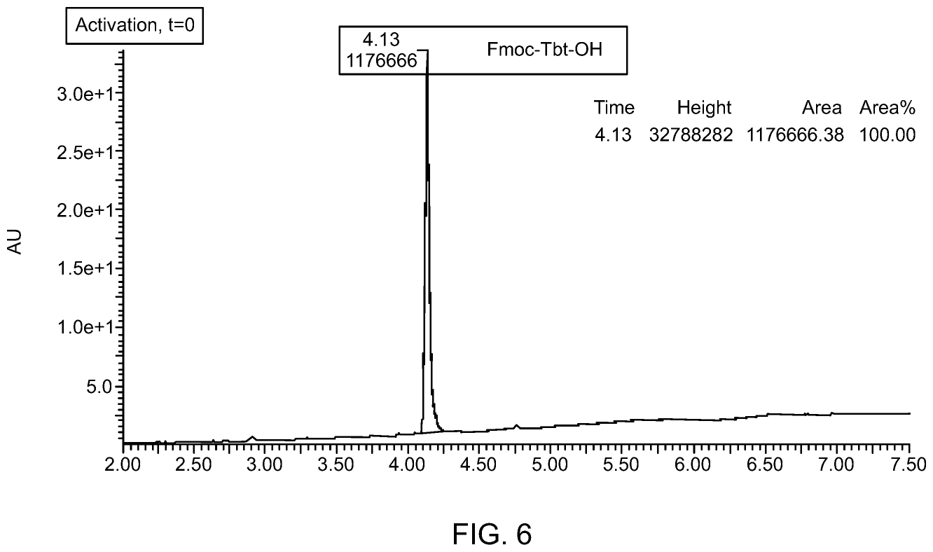

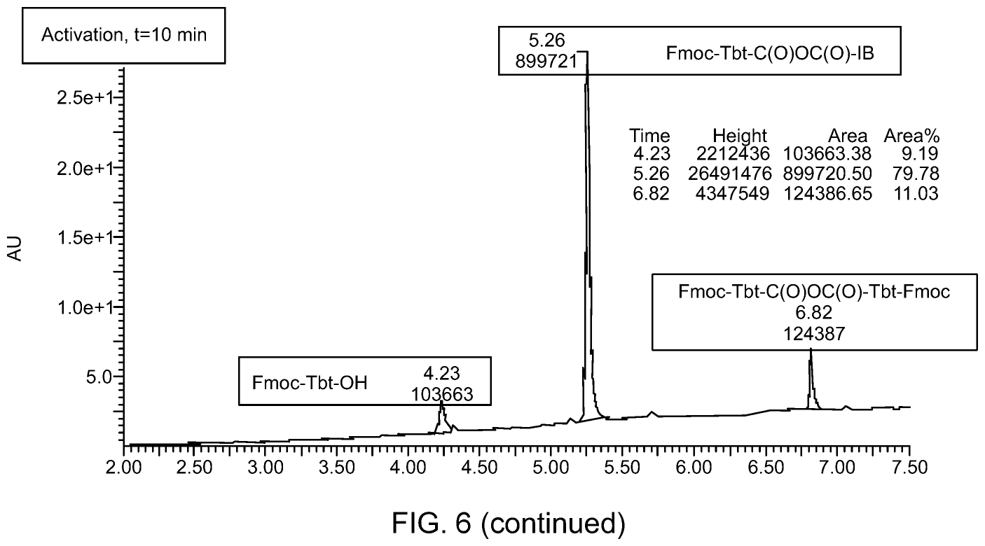

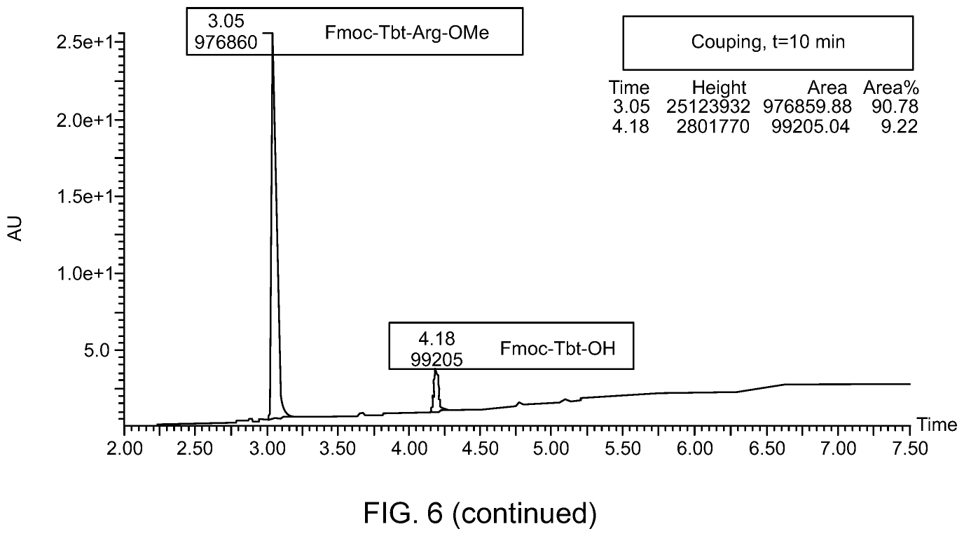

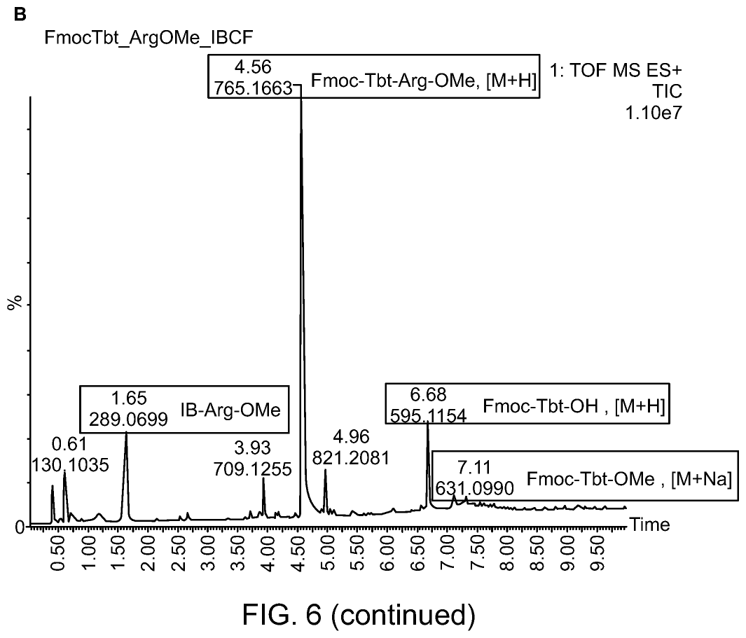

FIG. 6 shows UPLC-PDA traces (FIG. 6A) of the activation and coupling of Fmoc-Tbt-OH to H-Arg-OMe using isobutylchloroformate and UPLC-MS spectra (FIG. 6B) after completion of the coupling reaction.

### DETAILED DESCRIPTION

The method of the invention is for making a target peptide and involves reacting a mixed anhydride compound of Formula (I) with a second moiety which is an amino acid or a peptide.

The reaction results in the formation of an amide bond between the carboxyl group of the Tbt residue in Formula (I) and the amino group of the second moiety. The reaction between the mixed anhydride compound of Formula (I) and the second moiety may form the target peptide or a precursor to the target peptide. In some cases, subsequent removal of protecting groups or further peptide coupling reactions may be required to convert the precursor into the target peptide.

As set out in the Summary of the Invention, one aspect of the invention is directed to a compound of Formula (I).

### Mixed Anhydride Compound of Formula (I)

Formula (I) comprises an extremely sterically bulky tri-tert-butyl-tryptophan (Tbt) residue and has the structure:

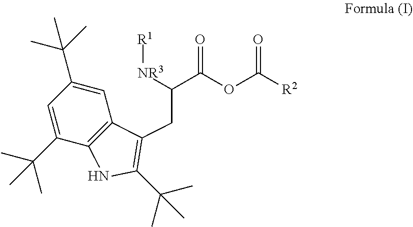

As illustrated in FIG. 1, there are two carbonyl groups in Formula (I) that an amino nucleophile in the second moiety could attack. Attack at the “correct” carbonyl (the Tbt carbonyl) results in the desired formation of the amide bond whereas attack at the “wrong” carbonyl (the carbonyl adjacent to R 2 which may be derived from the activator of Formula (III) as set out below) does not result in the formation of the amide bond.

The inventors have unexpectedly found that, in spite of the very high steric bulk of the Tbt side chain, the mixed anhydride of Formula (I) reacts with amino acids or peptides quickly, thereby reducing undesirable epimerization, and with good selectivity for the “correct” carbonyl (the Tbt carbonyl). In comparative experiments employing amino acids that are far less sterically hindered than Tbt, the coupling reactions were slow and significant amounts of product resulting from reaction at the “wrong” carbonyl (the carbonyl adjacent to R²) were observed.

In Formula (I), R¹ is: a protecting group, a peptide or an amino acid. Optionally, the peptide or the amino acid may themselves comprise one or more protecting groups, such as on their N-terminal amino groups.

As used herein the term peptide includes peptomimetics, although true peptides are preferred. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the target peptide reacts with a membrane rather than the specific active site of an enzyme, some of the problems described of exactly mimicing affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The term ‘amino acid’ may thus conveniently be used herein to refer to the equivalent sub-units of a peptidomimetic compound. Moreover, peptidomimetics may have groups equivalent to the R groups of amino acids.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen. Preferred peptidomimetic backbones include polyesters, polyamines and derivatives thereof as well as substituted alkanes and alkenes. The peptidomimetics will preferably have N and C terminii which may be modified as discussed herein.

Preferably, the term “amino acid” as used herein refers to proteinogenic (genetically encoded) amino acids. Preferably, the term peptide in R¹ and the second moiety refers to a peptide formed from proteinogenic amino acids.

R¹ typically comprises 1 to 10 amino acids, preferably 1 to 5 or 1 to 3 amino acids, most preferably 1 amino acid.

A wide choice of protecting groups suitable for amino acids are known (see, for example, Greene, T. W. and Wuts, P. G. M., Protective Groups in Organic Synthesis, 3rd ed., Wiley: New York, 1999 and Isidro-Llobet et al., Chem. Rev. 2009, 109, 6, 2455-2504).

Suitable amine protecting groups include carbobenzoxy (also known as benzyloxycarbonyl and designated Z or Cbz), t-butoxycarbonyl (also designated Boc), 4-methoxy-2,3,6-trimethylbenzene sulphonyl (Mtr), 9-fluorenylmethoxycarbonyl (also designated Fmoc) and 2,2,2-trichloroethoxycarbonyl (Troc). These protecting groups may themselves be R¹ or one or more of these protecting groups may be present on R¹ when R¹ is a peptide or an amino acid.

Suitable carboxyl protecting groups which may, for example be employed include readily cleaved ester groups such as benzyl (Bn), p-nitrobenzyl (pNb), pentachlorophenyl (PCIP), pentafluorophenyl (Pfp) or t-butyl (tBu) groups as well as the coupling groups on solid supports, for example methyl groups linked to polystyrene. Other suitable carboxyl protecting groups include 4-{N-[1-(4,4-dimethyl-2,6-dioxocyclohexylidene)-3-methylbutyl]amino}benzyl ester (Dmab), allyloxycarbonyl (Alloc) and 2-phenylisopropyl (2-PhiPr).

Thiol protecting groups include p-methoxybenzyl (Mob), trityl (Trt), acetamidomethyl (Acm), tert-butyl (tBu), tert-butylthio (tButhio) and monomethoxytrityl (Mmt) groups.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

Preferably, R¹ is a peptide or an amino acid, optionally comprising one or more protecting groups, such as on its N-terminal amino group. The amino acid may be a cationic amino acid AA₁.

Preferably, R¹ is a cationic amino acid AA₁ optionally comprising one or more protecting groups, such as on its N-terminal amino group.

AA₁ is preferably, lysine or arginine but may be histidine or any non genetically coded or modified amino acid carrying a positive charge at pH 7.0. Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Most preferably, R¹ is arginine optionally comprising one or more protecting groups, such as on its N-terminal amino group.

In Formula (I), R² is selected from tert-butyl, isobutoxy, tert-butoxy, isobutyl, isopropoxy, isopropyl, or ethoxy. Preferably, R² is selected from tert-butyl, isobutoxy, tert-butoxy, isobutyl, isopropoxy, or isopropyl. More preferably, R² is tert-butyl or isobutoxy, most preferably R² is tert-butyl.

In other preferred cases, R² is one of the alkyl groups defined above, i.e. tert-butyl, isobutyl or isopropyl, preferably tert-butyl.

In Formula (I), R³ is selected from H or an alkylsilyl group, such as a mono(C₁-C₆ alkyl)silyl, di(C₁-C₆ alkyl)silyl or tri(C₁-C₆ alkyl)silyl group. Preferably, the alkylsilyl group is a tri(C₁-C₆ alkyl)silyl group, more preferably a tri(C₁-C₃ alkyl)silyl group. Each alkyl group may be the same or different, preferably the same. If present as R³, the alkylsilyl group is preferably trimethyl silyl.

R³ is preferably H. That is, the compound of Formula (I) preferably has the structure

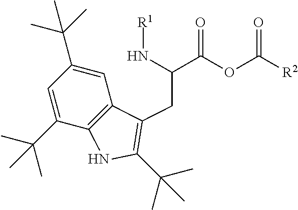

### Second Moiety

The second moiety is an amino acid or a peptide. The amino acid or peptide of the second moiety may optionally contain one or more protecting groups and/or a C-terminal capping group. The amino acid or peptide of the second moiety may optionally be silylated. Suitable silylating agents are disclosed in WO 2009/065836. For example, suitable silylating agents are N-trialkylsilyl amines or N-trialkylsilyl amides, such as those selected from the group consisting of: N,O-bis(trimethylsilypacetamide, N,O-bis(trimethylsily0trifluoroacetamide, hexamethyldisilazane, N-methyl-N-trimethylsilylacetamide (MSA), N-methyl-N-trimethylsilyltrifluoroacetamide, N-(trimethylsilyl)acetamide, N-(trimethylsilyl)diethylamine, N-(trimethylsilyl)dimethylamine, 1-(trimethylsilyl)imidazole and 3-(trimethylsilyl)-2-oxazolidone. Silylation may improve solubility of the second moiety, for example in polar organic solvents such as polar aprotic solvents, e.g. dimethylacetamide. During silylation one or more functional groups in the second moiety having an active hydrogen, such as amino, hydroxyl, mercapto or carboxyl groups, react with the silylating agent. The silylated second moiety then comprises one or more silyl groups (such as trialkylsilyl, typically tri(C₁-C₃)alkyl groups such as trimethylsilyl) bonded to said functional groups. The amino acid or peptide of the second moiety may optionally be provided in the form of a salt, such as a hydrochloride, trifluoroacetate or acetate salt.

The second moiety typically comprises a reactive amino group, in particular an alpha-amino group. The alpha-amino group is preferably not protonated so that it is available to act as a nucleophile.

The second moiety typically comprises 1 to 10 amino acids, preferably 1 to 5 or 1 to 3 amino acids, most preferably 1 amino acid.

As set out above, suitable protecting groups for amino acids are well known and the protecting groups listed above may also be used in the second moiety.

Suitable C-terminal capping groups are of formula —X—Y—Z, wherein the left hyphen denotes the point of attachment to the carbon of the C-terminal carbonyl and X, Y and Z are defined as for Formula (IV) below. In other words, if present, capping group —X—Y—Z is attached to the remainder of the second moiety as follows:

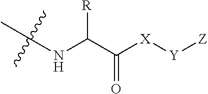

wherein R denotes the side chain of the C-terminal amino acid. Preferably, —X—Y—Z together is the group —NHCH₂CH₂Ph.

Preferably, the second moiety is a compound of Formula (IV):

AA₃-X—Y—Z (IV)
wherein AA₃ is a cationic amino acid, preferably lysine or arginine but may be histidine or any non genetically coded or modified amino acid carrying a positive charge at pH 7.0;
X is a N atom, which may be, but preferably is not, substituted by a branched or unbranched C₁-C₁₀ alkyl or aryl group (such as a C₄-C₁₀ aryl group), e.g. methyl, ethyl or phenyl, and this alkyl or aryl group may incorporate up to 2 heteroatoms selected from N, O and S;
Y represents a group selected from —Rₐ—R_{b}—, —Rₐ—R_{b}—R_{b}— and —R_{b}—R_{b}—Rₐ— wherein
Rₐ is C, O, S or N, preferably C, and
R_{b} is C; each of Rₐ and R_{b} may be substituted by C₁-C₄ alkyl groups or unsubstituted, preferably Y is —Rₐ—R_{b}— (in which Rₐ is preferably C) and preferably this group is not substituted, when Y is —Rₐ—R_{b}—R_{c}— or R_{b}—R_{b}—Rₐ— then preferably one or more of Rₐ and R_{b} is substituted; and
Z is a group comprising 1 to 3 cyclic groups each of 5 or 6 non-hydrogen atoms (preferably C atoms), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; the Z moiety incorporates a maximum of 15 non-hydrogen atoms, preferably 5-12, most preferably it is phenyl;
   the bond between Y and Z is a covalent bond between Rₐ or R_{b} of Y and a non-hydrogen atom of one of the cyclic groups of Z.

The compound of Formula (IV) may optionally contain one or more protecting groups and/or be silylated. The discussion of silylation and suitable silylating agents above applies equally when the second moiety is a compound of Formula (IV).

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Preferably, Y is —Rₐ—R_{b}— as defined above, more preferably wherein Rₐ and R_{b} are unsubstituted, most preferably wherein Rₐ and R_{b} are both carbon atoms. In other words, Y is most preferably —CH₂CH₂—.

Preferably, —X—Y—Z together is the group —NHCH₂CH₂Ph.

Most preferably, AA₃ is arginine.

In other preferred cases, the second moiety is an amino acid comprising AA₃ or a peptide comprising AA₃ as the N-terminal amino acid, optionally wherein the amino acid or peptide comprise one or more protecting groups and/or a C-terminal capping group. The definitions of AA₃ above in the context of Formula (IV) apply equally in this case. The C-terminal capping group may have the structure —X—Y—Z, and the preferred definitions of each of —X—Y—Z set out above also apply equally to this case. The amino acid comprising AA₃ or the peptide comprising AA₃ as the N-terminal amino acid may optionally be silylated, and the discussion of silylation and suitable silylating agents above applies equally.

The compounds of the invention and those used and made in/by the methods of the invention (e.g. of Formula (I), the target peptide and the second moiety) may include all enantiomeric forms, both D and L amino acids and enantiomers resulting from chiral centers within the amino acid R groups and moieties Y or Z.

More preferably, the second moiety is arginine, optionally comprising one or more protecting groups and/or a C-terminal capping group. In this case, the C-terminal capping group may be of formula —X—Y—Z, and is preferably —NHCH₂CH₂Ph.

The reaction between the compound of Formula (I) and the second moiety may be carried out in a suitable solvent. Suitable solvents include aqueous or non-aqueous solvents. Examples of suitable solvents include water; DCM; DMF; methanol; N,N-dimethylacetamide (DMA); acetonitrile (ACN); N-methylpyrrolidone and dimethylsulfoxide; 2-methyltetrahydrofuran (MeTHF) or a mixture thereof, such as a mixture of DMF and methanol. Preferably, the solvent is DMF or a mixture of DMF and methanol. Other preferred solvents include ACN, MeTHF, DMA and mixtures thereof.

The second moiety is preferably added to the compound of Formula (I) in the form of a solution. In some preferred cases, the second moiety may be added to the compound of Formula (I) in the form of a solution comprising DMA solvent.

Prior to the reaction with the compound of Formula (I), the second moiety may optionally be treated with an acid to improve solubility, such as in DMA solvent. Any suitable acid may be used. For example, suitable acids include hydrochloric acid, trifluoroacetic acid, acetic acid. The acid may be provided in aqueous or non-aqueous form. Optionally, approximately 3 equivalents of acid per equivalent of the second moiety may be added, such as from 2.5 to 3.5 equivalents or from 2.9 to 3.3 equivalents of the acid per equivalent of second moiety.

Surprisingly the reaction is fast despite the very high steric bulk of the Tbt side chain, which may reduce epimerization and thereby facilitate purification.

The reaction may be carried out at room temperature and pressure, e.g. 20-25° C. and 1 atmosphere of pressure. The reaction may also be carried out at lower temperatures. For example, the reaction may be carried out at any temperature from approximately −20° C. to room temperature, such as from −15° C. to 25° C., preferably from −10° C. to 20° C.

The reaction between the compound of Formula (I) and the second moiety may optionally be carried out in the presence of a base. Optionally, one or more bases may be used. The use of a base may ensure that the α-amino group is not protonated. Thus, the base(s) may be added in an amount sufficient to ensure that the α-amino group is not protonated so that it is available to act as a nucleophile. For example, one molar equivalent of base per molar equivalent of second moiety may be used. Where the second moiety has previously been treated with an acid as set out above, more than one equivalent of base may be required. For example, when the second moiety comprises an amino acid having a basic side group and has been treated with approximately 3 equivalents of acid per equivalent of second moiety (such as from 2.5 to 3.5 equivalents or from 2.9 to 3.3 equivalents of acid per equivalent of second moiety), approximately 2 equivalents of acid (such as from 1.5-2.5 or 1.8-2.2 equivalents of base) per equivalent of second moiety may be used. Suitable bases include: DIPEA, N-methylmorpholine, pyridine, trimethylamine and 2,4,6-trimethylpyridine. Preferably, the base is DIPEA. If used, the base may preferably be added after mixing the second moiety and the compound of Formula (I).

The method of the invention may further comprise one or more purification steps, such as to remove side products resulting from the reaction of the second moiety at the “wrong” carbonyl of Formula (I).

### Tbt-Activation and Activator

The mixed anhydride compound of Formula (I) may be prepared by reacting a first moiety of Formula (II) with an activator of Formula (III) in the presence of a base. Thus, in one aspect, the method of the invention comprises first preparing the compound of Formula (I) by reacting a first moiety of Formula (II) with an activator of Formula (III) in the presence of a base.

The compound of Formula (I) may optionally be isolated before reaction with the second moiety. Preferably, the compound of Formula (I) is not isolated before reaction with the second moiety.

Formula (II) has the structure:

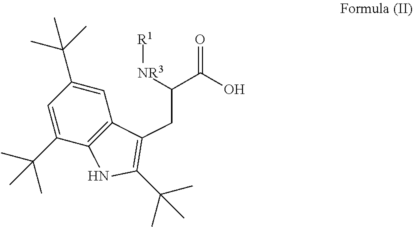

wherein R¹ is a protecting group, a peptide or an amino acid. The discussion of R¹ and R³ above in connection with Formula (I) applies equally to Formula (II). For example, R³ is preferably H and so Formula (II) preferably has the structure

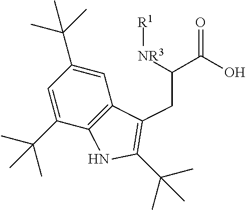

Formula (III) has the structure:

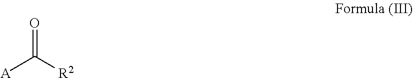

wherein R² is tert-butyl, isobutoxy, tert-butoxy, isobutyl, isopropoxy, isopropyl, or ethoxy. Preferably, R² is selected from tert-butyl, isobutoxy, tert-butoxy, isobutyl, isopropoxy, or isopropyl. More preferably, R² is tert-butyl or isobutoxy, most preferably R² is tert-butyl.

In other preferred cases, R² is one of the alkyl groups defined above, i.e. tert-butyl, isobutyl or isopropyl, preferably tert-butyl.

A is a halogen, such as Cl, Br or I. Preferably, A is Cl.

The activator of Formula (III) is preferably pivaloyl chloride or isobutylchloroformate, more preferably, pivaloyl chloride.

Any suitable base may be used, for example suitable bases include N,N-diisopropylethylamine (DIPEA), N-methylmorpholine, pyridine, and triethylamine. Preferably, the base is N,N-diisopropylethylamine (DIPEA).

The reaction between the compounds of Formula (II) and Formula (III) may be carried out in a non-aqueous solvent. Suitable solvents include DCM; DMF; N,N-dimethylacetamide; acetonitrile (ACN); N-methylpyrrolidone and dimethylsulfoxide; MeTHF; or a mixture thereof. Preferably, the solvent is DMF. In other preferred cases, the solvent is a mixture of ACN and MeTHF.

The reaction between the compounds of Formula (II) and Formula (III) may be carried out at room temperature and pressure, e.g. 20-25° C. and 1 atmosphere of pressure. The reaction may also be carried out at lower temperatures. For example, the reaction may be carried out at any temperature from approximately −20° C. to room temperature, such as from −15° C. to 25° C., preferably from −10° C. to 20° C.

The method of the invention may further comprise preparing the compound of Formula (II). For example, when R¹ is a peptide or an amino acid, the method may further comprise coupling said peptide or amino acid to the Tbt residue. Any suitable peptide coupling technique may be used to link the R¹ group to the Tbt residue. In some cases, the compound of Formula (II) may be prepared by pre-activating the R¹ amino acid or peptide, such as with pivaloyl chloride or isobutylchloroformate, and then coupling with Tbt, optionally silylated Tbt. Peptide production methods involving silylated peptides are disclosed in WO2009/065836.

The method of the invention may also further comprise preparing the second moiety. For example, when the second moiety comprises the C-terminal capping group, such as a capping group having formula —X—Y—Z as defined above (e.g. —NHCH₂CH₂Ph), the process may comprise activating the C-terminal carboxylic acid group of the second moiety, which typically comprises an amino protecting group such as Cbz, and then coupling to H—X—Y—Z (e.g. H₂NCH₂CH₂Ph). Suitable activators for this step include pivaloyl chloride or isobutylchloroformate.

### Target Peptide

A target peptide according to the present invention will typically have a chain length of up to 20 amino acids. Preferably, target peptides are 2 to 10, 3 to 7 or 3 to 5, e.g. 3 amino acids in length.

The target peptide is preferably an antimicrobial peptide.

Preferably, the target peptide is a compound of Formula (V)

AA₁-AA₂-AA₃-X—Y—Z (V)
wherein:
each AA₁ and AA₃ is independently a cationic amino acid, preferably lysine or arginine but may be histidine or any non-genetically coded or modified amino acid carrying a positive charge at pH 7.0;
AA₂ is Tbt, i.e.

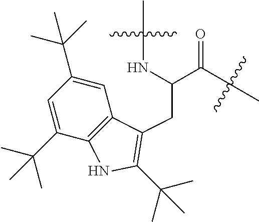 [SMILES: *NC(Cc1c(C(C)(C)C)[nH]c2c(C(C)(C)C)cc(C(C)(C)C)cc12)C(*)=O]

wherein the left hand wiggly bond denotes the point of attachment to AA₁ and the right hand wiggly bond denotes the point of attachment to AA₃-X—Y—Z; and
X, Y and Z are as defined above.

Non-genetically coded or modified amino acids that are suitable as AA₁ and/or AA₃ are set out above.
The compounds of Formula (V) are antimicrobial peptides and are disclosed in WO2009/081152.

The target peptide may include all enantiomeric forms, both D and L amino acids and enantiomers resulting from chiral centers within the amino acid R groups and moieties Y or Z, when present.

Preferably, the target peptide is Arg-Tbt-Arg-NHCH₂CH₂Ph, i.e. the compound

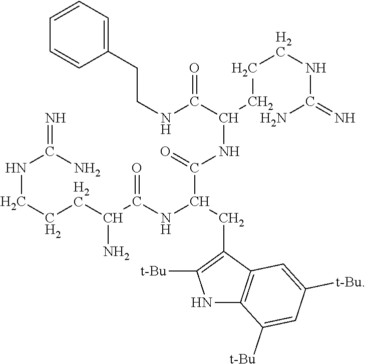 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

Most preferably, the target peptide has the following structure:

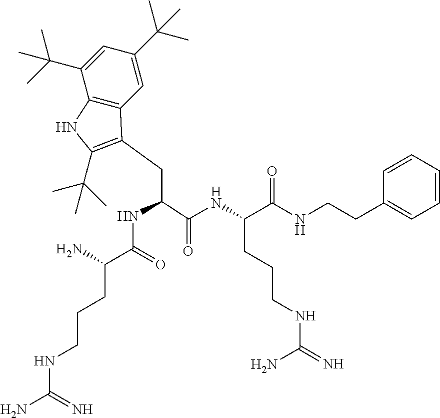 [SMILES: CC(C)(C)c1cc(C(C)(C)C)c2[nH]c(C(C)(C)C)c(C[C@H](NC(=O)[C@@H](N)CCCNC(=N)N)C(=O)N[C@@H](CCCNC(=N)N)C(=O)NCCc3ccccc3)c2c1]

which is also referred to herein as AMC-109.

Scheme 1 shows an exemplary synthesis strategy for the production of AMC-109 according to the invention.

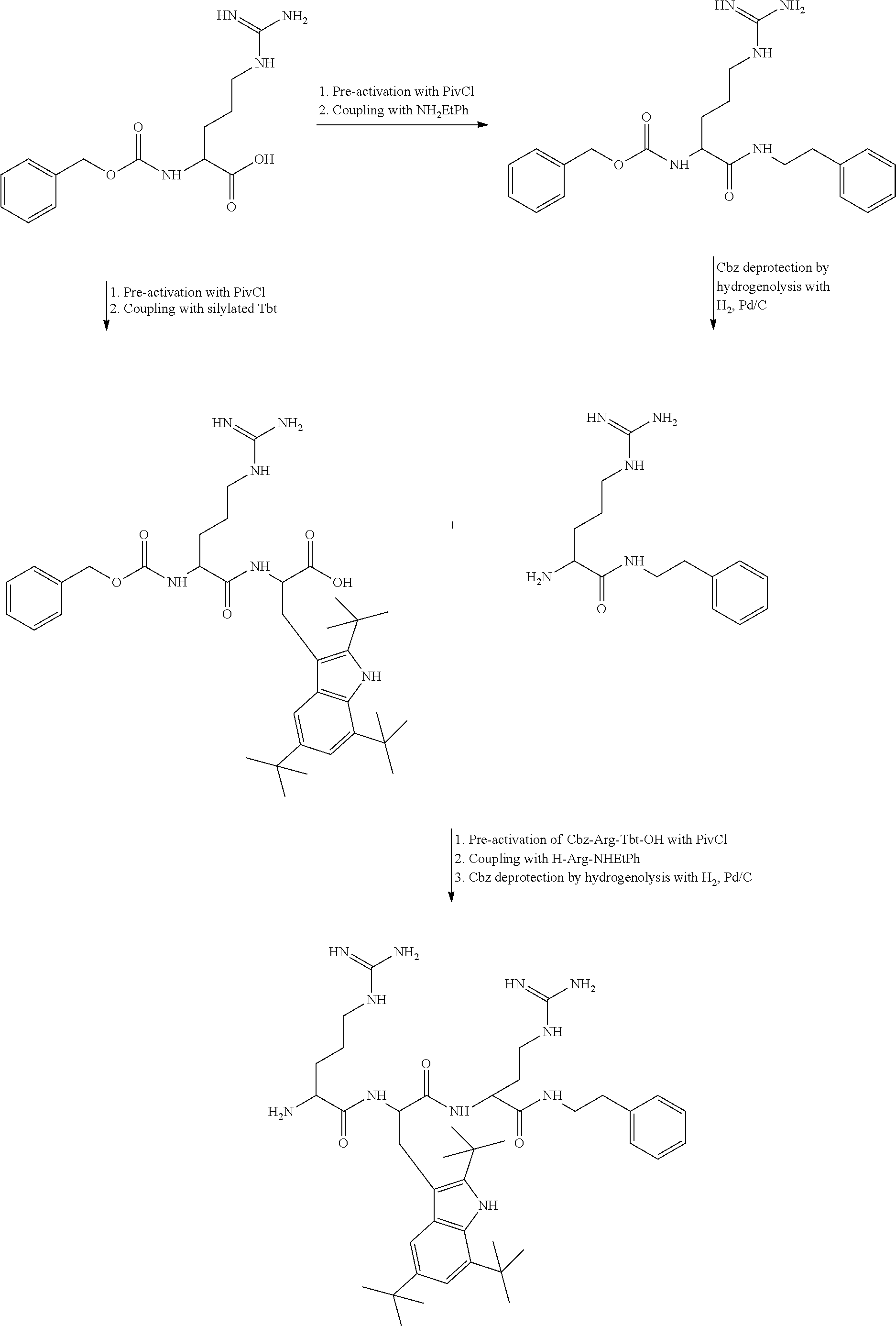 [SMILES: N=C(N)NCCCC(NC(=O)OCc1ccccc1)C(=O)O ; N=C(N)NCCCC(NC(=O)OCc1ccccc1)C(=O)NCCc1ccccc1 ; CC(C)(C)c1cc(C(C)(C)C)c2[nH]c(C(C)(C)C)c(CC(NC(=O)C(CCCNC(=N)N)NC(=O)OCc3ccccc3)C(=O)O)c2c1 ; N=C(N)NCCCC(N)C(=O)NCCc1ccccc1 ; CC(C)(C)c1cc(C(C)(C)C)c2[nH]c(C(C)(C)C)c(CC(NC(=O)C(N)CCCNC(=N)N)C(=O)NC(CCNC(=N)N)C(=O)NCCc3ccccc3)c2c1]

As illustrated in Scheme 1, the method of the invention may comprise steps of removing any protecting groups. For example, Cbz protecting groups may be removed by hydrogenolysis with H₂ over palladium on carbon (Pd/C).

### EXAMPLES

### Materials/Methods for Examples 1-3

Amino acid derivatives used

| | | |
|---|---|---|
| 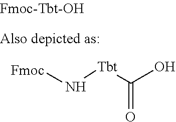 | 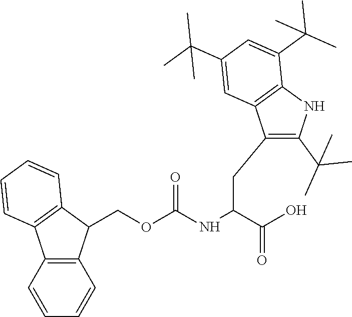 [SMILES: CC(C)(C)c1cc(C(C)(C)C)c2[nH]c(C(C)(C)C)c(CC(NC(=O)OCC3c4ccccc4-c4ccccc43)C(=O)O)c2c1] | MW = 594.35 g/mol |
| 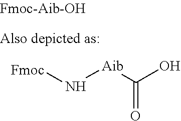 | 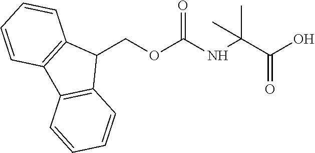 [SMILES: CC(C)(NC(=O)OCC1c2ccccc2-c2ccccc21)C(=O)O] | MW = 325.13 g/mol |
| Fmoc-Tle-OH | 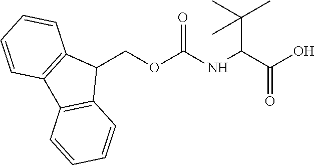 [SMILES: CC(C)(C)C(NC(=O)OCC1c2ccccc2-c2ccccc21)C(=O)O] | MW = 353.16 g/mol |
| 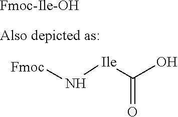 | 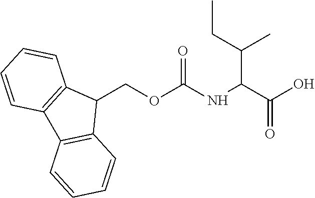 [SMILES: CCC(C)C(NC(=O)OCC1c2ccccc2-c2ccccc21)C(=O)O] | MW = 353.16 g/mol |
| H-Arg-OMe•2HCl ** | 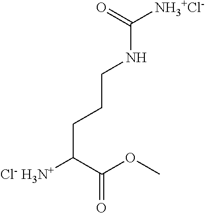 | MW = 261 g/mol |
| Cbz-Arg-OH•HCl | 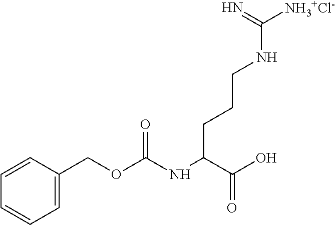 | MW = 344.80 g/mol |

** H-Arg-OMe is also depicted as H₂N-Arg-C(O)OMe below

The amino acid derivatives are commercially available.

### Reagents Used

| | |
|---|---|
| Diisopropylethylamine(DIPEA) | MW = 129 g/mole; |
| | d = 0.76 g/mL |
| O-(Benzotriazol-1-yl)-N,N,N′N′-tetramethyluronium | MW = 379 g/mole |
| hexafluorophospate (HBTU) | |
| Pivaloyl chloride (Piv-Cl) | MW = 121 g/mole; |
| g/mL | d = 0.98 |
| Oxalyl chloride (Oxa-Cl) | MW = 127 g/mole; |
| g/mL | d = 1.50 |
| Cyanuric chloride (Cya-Cl) | MW = 184 g/mole; |
| g/mL | d = 1.32 |
| Isobutyl chloroformate (IBCF) | MW = 137 g/mole; |
| g/mL | d = 1.05 |

### Solvents Used

| | |
|---|---|
| Dichloromethane (DCM) | Anhydrous, kept |
| sieves | on 3 Å molecular |
| Dimethylformamide (DMF) | Anhydrous, kept |
| sieves | on 3 Å molecular |
| Methanol (MeOH) | Anhydrous, kept |
| sieves | on 3 Å molecular |

### Monitoring of Reactions.

725 μL solv-B was transferred to an UPLC vial. A 25 μL reaction sample was added. The analyses were done on UPLC (see setup below) and the correct MW were confirmed using ESI-MS. The injection volume before the addition of H-Arg-OMe was 3 μL, after addition the injection volume was increased to 4 μL.

### UPLC-PDA Setup

System: Waters Acquity H-class UPLC
Column: Acquity UPLC BEH C18, 2.1×50 mm, with 1.7 μm particles
Detection: PDA (210-500 nm)
Mobile phases: (Solv-A) MilliQ water with 0.1% TFA; (Solv-B) Acetonitrile with 0.1% TFA
Flow rate: 0.6 mL/min
Gradients:

Gradient for Fmoc—Tbt

| Time (min) | A (%) | B (%) |
|---|---|---|
| 0.0 | 50 | 50 |
| 0.5 | 50 | 50 |
| 5.5 | 0 | 100 |
| 8.0 | 0 | 100 |
| 8.1 | 50 | 50 |
| 10.0 | 50 | 50 |

Gradient for Fmoc-Aib, Tle, and Ile

| Time (min) | A (%) | B (%) |
|---|---|---|
| 0.0 | 80 | 20 |
| 0.5 | 80 | 20 |
| 5.5 | 30 | 70 |
| 8.0 | 0 | 100 |
| 8.1 | 80 | 20 |
| 10.0 | 80 | 20 |

### UPLC-MS Setup

System: Waters Acuity I-class UPLC
Column: Acquity UPLC BEH C18, 2.1×50 mm, with 1.7 μm particles
Detection: Waters XEVO Q-ToF G2 mass spectrometer Tune method set at capillary 0.6 kV, sampling cone 30 V, source temperature 130° C., desolvation temperature 450° C., cone gas flow 10.0 L/h and desolvation gas flow 800.0 L/h
Mobile phases: (Solv-A) MilliQ water with 0.1% FA; (Solv-B) Acetonitrile with 0.1% FA
Flow rate: 0.5 mL/min

### Example 1 (Comparative)

### 1.1 Activation of Compound Comprising a Moderately Sterically Hindered Amino Acid 2-Aminoisobutyric Acid (Aib) Using Pivaloyl Chloride

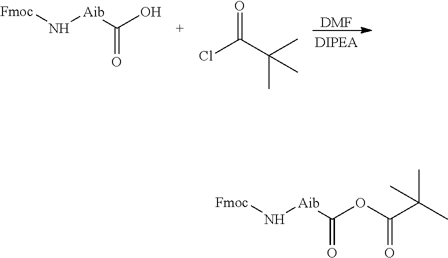

### Procedure

(i) In a small (10 mL) glass vial 3.25 mg Fmoc-Aib-OH (10 μmole) was weighed out and dissolved in 1.5 mL anhydrous DMF and 3.4 μL DIPEA (20 μmole, 2 eq) was added.

(ii) The reaction was started by adding 2.5 μL Piv-Cl (20 μmole, 2 eq).

(ii) Every 10 minutes a small sample was taken for UPLC-PDA analysis.

(iv) The total reaction time was 20 min.

### Result

Preactivation was completed in <10 min. A small amount (ca. 5%) of, presumably, a symmetrical anhydride of Fmoc-Aib-OH was formed, see FIG. 2A.

1.2 Coupling Reaction of Fmoc-Aib-C(O)OC(O)-Piv with H-Arg-OMe·2HCl

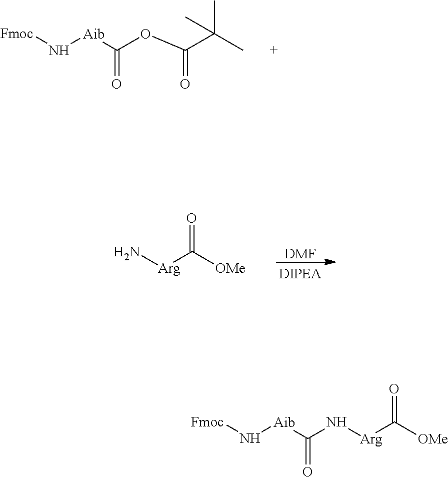

### Procedure

(i) In a small (10 mL) glass vial, 78 mg H-Arg-OMe·2HCl (300 μmole) was weighed out and dissolved in 5 mL anhydrous MeOH and 51 μL DIPEA (300 μmole, 1 eq) added.

(ii) To the pre-activation solution from reaction 1.1, 500 μL of the H-Arg-OMe·2HCl (30 μmole, 3 eq) solution was added.

(iii) Every 10 minutes a small sample was taken for UPLC-PDA analysis. At the completion of the reaction, the reaction mixture was analyzed using UPLC-MS.

### Result

The coupling reaction is slow and is only completed after 30 min. A significant amount of unwanted side reaction of the mixed anhydride intermediate was observed, resulting in only 20% of the desired Fmoc-Aib-Arg-OMe forming, see FIGS. 2A and B.

### 1.3 Activation of Compound Comprising a Moderately Sterically Hindered Amino Acid α-Tert-Butylglycine (Tle) Using Pivaloyl Chloride

### Procedure

(i) In a small (10 mL) glass vial 3.53 mg Fmoc-Tle-OH (10 μmole) was weighed out and dissolved in 1.5 mL anhydrous DMF and 3.4 μL DIPEA (20 μmole, 2 eq) added.

(ii) The reaction was started by adding 2.5 μL Piv-Cl (20 μmole, 2 eq).

(ii) Every 10 minutes a small sample was taken for UPLC-PDA analysis.

(iv) The total reaction time was 20 min.

### Result

Preactivation is completed in <10 min. No symmetrical anhydride of Fmoc-Tle-OH was observed, see FIG. 3A.

1.4 Coupling Reaction of Fmoc-Tle-C(O)OC(O)-Piv with H-Arg-OMe·2HCl

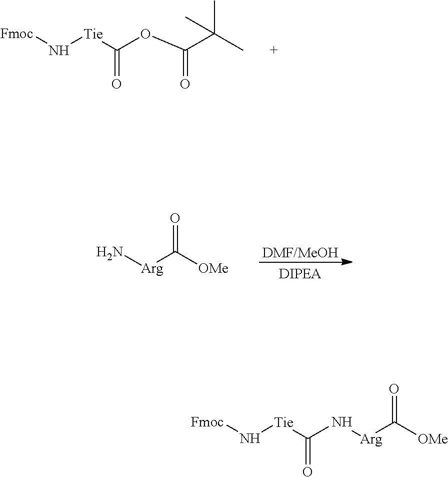

### Procedure

(i) In a small (10 mL) glass vial, 78 mg H-Arg-OMe·2HCl (300 μmole) was weighed out and dissolved in 5 mL anhydrous MeOH and 51 μL DIPEA (300 μmole, 1 eq) added.

(ii) To the pre-activation solution from reaction 1.3, 500 μL of the H-Arg-OMe·2HCl (30 μmole, 3 eq) solution was added.

(iii) Every 10 minutes a small sample was taken for UPLC-PDA analysis. At the completion of the reaction, the reaction mixture was analyzed using UPLC-MS.

### Result

The coupling reaction is slow and is only completed after 30 min. A significant amount of unwanted side reaction of the mixed anhydride intermediate was observed, resulting in only 40% of the desired Fmoc-Tle-Arg-OMe forming, see FIG. 3B.

### 1.5 Activation of Compound Comprising a Moderately Sterically Hindered Amino Acid Isoleucine (Ile) Using Pivaloyl Chloride

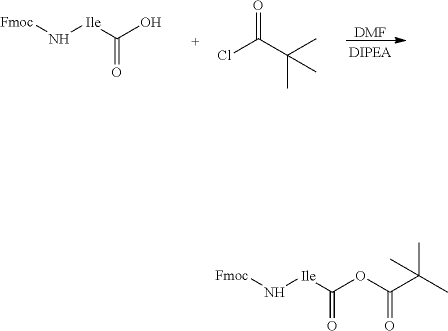

### Procedure

(i) In a small (10 mL) glass vial 3.53 mg Fmoc-Ile-OH (10 μmole) was weighed out and dissolved in 1.5 mL anhydrous DMF and 3.4 μL DIPEA (20 μmole, 2 eq) added.

(ii) The reaction was started by adding 2.5 μL Piv-Cl (20 μmole, 2 eq).

(ii) Every 10 minutes a small sample was taken for UPLC-PDA analysis.

(iv) The total reaction time was 20 min.

### Result

Preactivation is completed in <10 min. No symmetrical anhydride of Fmoc-Ile-OH was observed, see FIG. 4A.

1.6 Coupling Reaction of Fmoc-Ile-C(O)OC(O)-Piv with H-Arg-OMe·2HCl

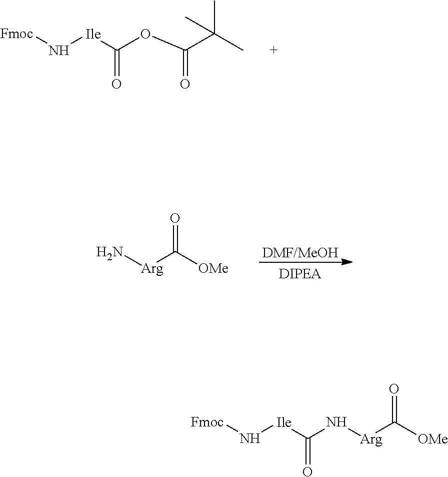

### Procedure

(i) In a small (10 mL) glass vial, 78 mg H-Arg-OMe·2HCl (300 μmole) was weighed out and dissolved in 5 mL anhydrous MeOH and 51 μL DIPEA (300 μmole, 1 eq) added.

(ii) To the pre-activation solution from reaction 1.5, 500 μL of the H-Arg-OMe·2HCl (30 μmole, 3 eq) solution was added.

(iii) Every 10 minutes a small sample was taken for UPLC-PDA analysis. At the completion of the reaction, the reaction mixture was analyzed using UPLC-MS.

### Result

The coupling reaction is relatively slow and is completed after 20 min. A significant amount of unwanted side reaction of the mixed anhydride intermediate was observed, resulting in only 37% of the desired Fmoc-Ile-Arg-OMe forming, see FIGS. 4A and 4B.

As shown in Example 1, a significant amount of unwanted side reaction of the mixed anhydride intermediates at the “wrong” carbonyl was observed for moderately sterically hindered amino acids containing Aib, Tle or Ile after activation with pivaloyl chloride.

### Example 2

### 2.1 Activation of Amino Acid Comprising Tri-Tert-Butyl-Tryptophan (Tbt) Using Pivaloyl Chloride

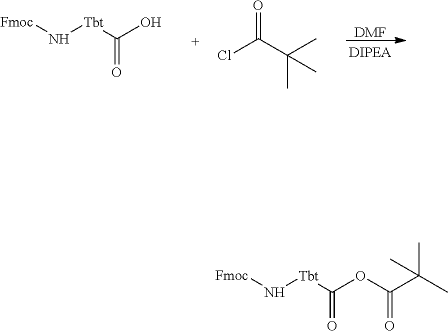

### Procedure

(i) In a small (10 mL) glass vial 5.95 mg Fmoc-Tbt-OH (10 μmole) was weighed out and dissolved in 1.5 mL anhydrous DMF and 3.4 μL DIPEA (20 μmole, 2 eq) added.

(ii) The reaction was started by adding 2.5 μL Piv-CI (20 μmole, 2 eq).

(ii) Every 10 minutes a small sample was taken for UPLC-PDA analysis.

(iv) The total reaction time was 20 min.

### Results

Preactivation is completed in less than 10 min, see FIG. 5A. The formed Fmoc-Tbt-C(O)OC(O)Piv is stable and no degradation was observed during the 60 min pre-activation period (results not shown). A small amount (ca. 2%) of, presumably, a symmetrical anhydride of Fmoc-Tbt-OH was formed. This reaction was also possible when using DCM as solvent (results not shown).

2.2 Coupling Reaction of Fmoc-Tbt-C(O)OC(O)-Piv with H-Arg-OMe·2HCl

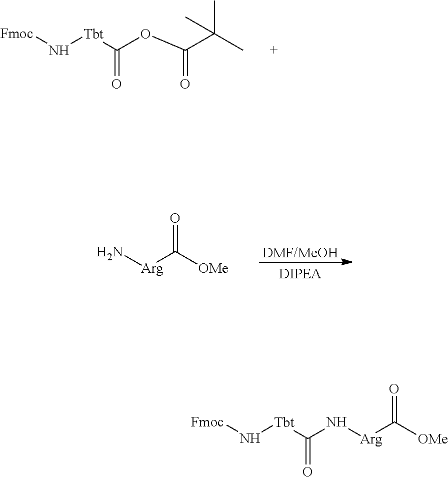

### Procedure

(i) In a small (10 mL) glass vial, 78 mg H-Arg-OMe·2HCl (300 μmole) was weighed out and dissolved in 5 mL anhydrous MeOH and 51 μL DIPEA (300 μmole, 1 eq) added.

(ii) To the pre-activation solution from reaction 2.1, 500 μL of the H-Arg-OMe·2HCl (30 μmole, 3 eq) solution was added.

(iii) Every 10 minutes a small sample was taken for UPLC-PDA analysis. At the completion of the reaction, the reaction mixture was analyzed using UPLC-MS.

### Results

The coupling reaction is unexpectedly fast and is completed <10 min. The main product is the desired Fmoc-Tbt-Arg-OMe, but there is a small amount (<9%) of Fmoc-Tbt-OH. This is due to the a-amine group of H-Arg-OMe attacking the wrong carbonyl of the formed mixed anhydride.

The presence of Piv-Arg-OMe was confirmed with UPLC-MS, see FIG. 5B. The reason for this is most likely due to the steric hinderance effect of Tbt's bulky side chain. To ensure that the reaction was between the α-amine group of H-Arg-OMe and not the guanidine group, the reaction was repeated using Cbz-Arg-OH·HCl. No reaction was observed (results not shown).

Although a small amount of unwanted side reaction of the mixed anhydride intermediate was observed when coupling the ultra-bulky Fmoc-Tbt-OH and H-Arg-OMe·2HCl, it was significantly less than that observed for the other, less bulky, amino acids Fmoc-Aib-OH, Fmoc-Tle-OH, and Fmoc-Ile-OH.

The coupling reactions were fast, with no signs of Tbt-epimerization or unwanted acylation through the unprotected guanidine side chain.

### Example 3

### 3.1 Activation of Amino Acid Comprising Tri-Tert-Butyl-Tryptophan (Tbt) Using Isobutyl Chloroformate (IBCF)

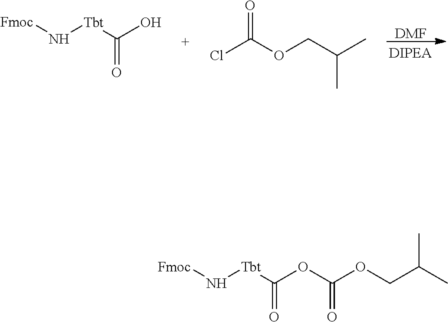

### Procedure

(i) In a small (10 mL) glass vial 5.95 mg Fmoc-Tbt-OH (10 μmole) was weighed out and dissolved in 1.5 mL anhydrous DMF and 3.4 μL DIPEA (20 μmole, 2 eq) added.

(ii) The reaction was started by adding 2.6 μL IBCF (20 μmole, 2 eq).

(ii) Every 10 minutes a small sample was taken for UPLC-PDA analysis.

(iv) The total reaction time was 20 min.

### Result

Preactivation is completed in <10 min. A significant amount (ca. 11%) of, presumably, a symmetrical anhydride of Fmoc-Tbt-OH was formed, see FIG. 6A.

3.2 Coupling Reaction of Fmoc-Tbt-C(O)OC(O)-IB with H-Arg-OMe·2HCl

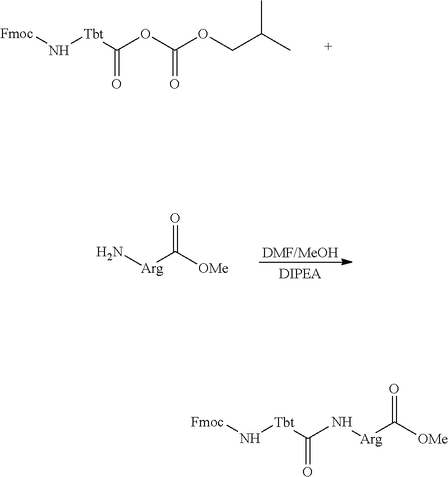

### Procedure

(i) In a small (10 mL) glass vial, 78 mg H-Arg-OMe·2HCl (300 μmole) was weighed out and dissolved in 5 mL anhydrous MeOH and 51 μL DIPEA (300 μmole, 1 eq) added.

(ii) To the pre-activation solution from reaction 3.1, 500 μL of the H-Arg-OMe·2HCl (30 μmole, 3 eq) solution was added.

(iii) Every 10 minutes a small sample was taken for UPLC-PDA analysis. At the completion of the reaction, the reaction mixture was analyzed using UPLC-MS.

### Result

The coupling reaction is fast and is completed <10 min. The main product is the desired Fmoc-Tbt-Arg-OMe, but there is also a small amount (ca. 9%) of Fmoc-Tbt-OH present (FIG. 6B).

### Example 4

### 4.1 Preparation of Intermediates Z-Arg-Tbt-OH (AMC-01) and H-Arg-NHEtPh (AMC-03)

Z-Arg-Tbt-OH (AMC-01) was prepared by activation of Cbz-protected arginine with IBCF followed by coupling with silylated Tbt as illustrated in the following scheme.

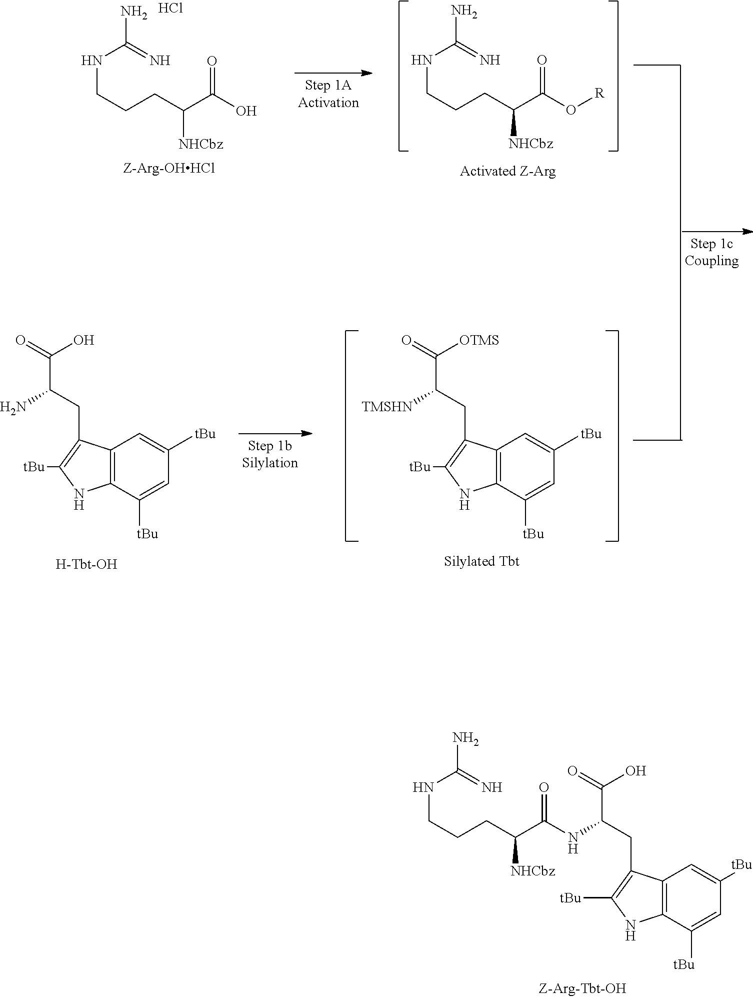

Z-Arg-NHEtPh (AMC-02) was prepared by activating commercially available Z-Arg-OH·HCl with IBCF, reacting activated Cbz-protected arginine with H₂NEtPh, and deprotection to provide H-ArgNHEtPh (AMC-03) as illustrated in the following schemes:

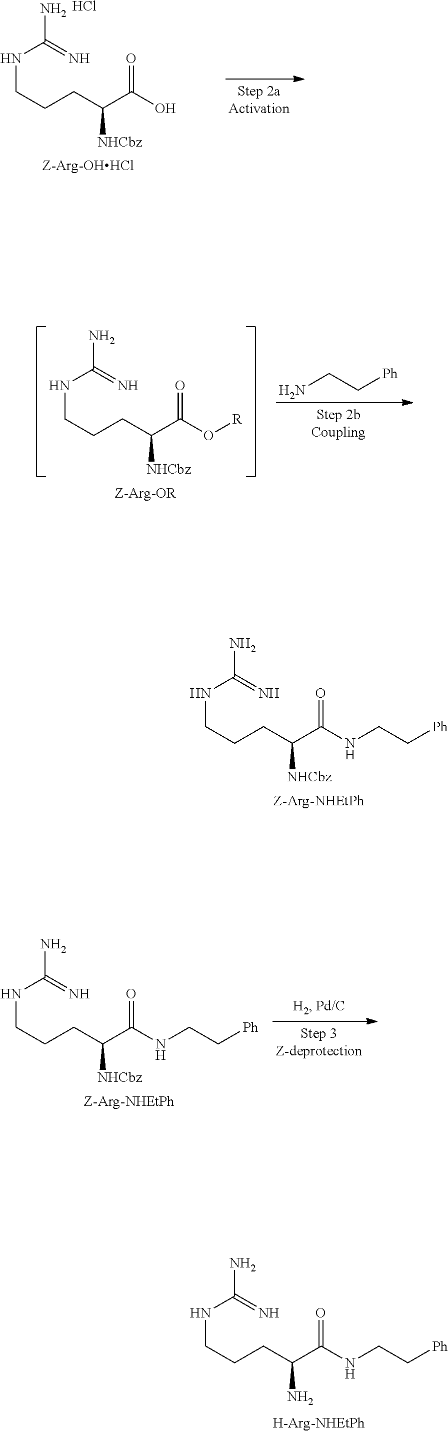

The procedure for the deprotection step (Step 3) was as follows. AMC-02 (37.20 g, 75% wt.), MeOH (550 mL) and water (130 mL) were introduced into a 2 L three neck flask. The suspension was stirred under nitrogen until all of the AMC-02 was fully soluble. Pd/C (10% wt, 50% wet, 2.27 g, 1.5 mol %) was added and the N₂ atmosphere was replaced with H₂ (using a H₂ generator, 0.7 bar). Deprotection conversion was followed by HPLC and full conversion was obtained after 3 h. Pd/C was then filtered, washed twice with MeOH/water (8/2, v/v, 2×75 mL). The filtrates were combined and concentrated under reduced pressure (Tbath=55° C., from 300 mbar to 50 mbar) up to 31 g of concentrated solution. DMA (100 mL) was added and evaporation was pursued (Tbath=65° C., 25 mbar) to afford AMC-03 as a colourless solution (117 mL, 115 g, 17% wt by NMR, est. 19.6 g net peptide, >99% yield).

### 4.2 Coupling of Z-Arg-Tbt-OH (AMC-01) and Z-Arg-NHEtPh (AMC-02) to Provide Z-Arg-Tbt-Arg-NHEtPh (AMC-04)

Activation of AMC-01 (1 eq) was carried out with PivCl (1.05 eq) and pyridine (1.05 eq) in a 2:1 v/v mixture of ACN/2-MeTHF at −10° C. for 15 minutes.

The colourless solution of AMC-03 was acidified with 2.5 N aqueous HCl (3.15 eq vs AMC-03). AMC-03 (1.05 eq) in the form of this acidified solution was then added to the activated AMC-01, followed by DIPEA (2, 10 eq vs AMC-03). The coupling reaction was conducted at −10° C. for 1 hour and progress of the reaction was followed by HPLC.

The parameters for the HPLC method are set out below:

| Column | Waters XSelect CSH C18 XP, 130 A, | | | | | |
|---|---|---|---|---|---|---|
| | 2.5 pm, 3 × 75 mm | | | | | |
| Oven | 40° C. | | | | | |
| Temperature | | | | | | |
| Wavelength | 220 nm | | | | | |
| Mobile | A: 0.1% TFA in water | | | | | |
| Phases | B: 0.1% TFA in ACN | | | | | |
| Flow Rate | 1 mL/min | | | | | |
| | | Time (min) | | | | |
| | | 0 | 7.5 | 8.1 | 8.2 | 10.7 |
| Gradient | % A | 98 | 0 | 0 | 98 | 98 |
| | % B | 2 | 100 | 100 | 2 | 2 |

### Result

The main product is the desired Z-Arg-Tbt-Arg-NHEtPh and only a small amount of AMC-01 (9%) was present.

1. A method for making a target peptide comprising reacting a mixed anhydride compound of Formula (I) with a second moiety which is an amino acid or peptide;
wherein Formula (I) has the structure: 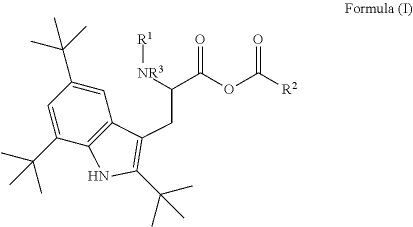
wherein R¹ is a protecting group, a peptide or an amino acid;
wherein R² is tent-butyl, isobutoxy, tert-butoxy, isobutyl, isopropoxy, isopropyl, or ethoxy; and
wherein R³ is H or an alkylsilyl group.

2. The method of claim 1 comprising preparing the mixed anhydride compound of Formula (I) by reacting a first moiety of Formula (II) with an activator of Formula (III) in the presence of a base;
wherein Formula (II) has the structure: 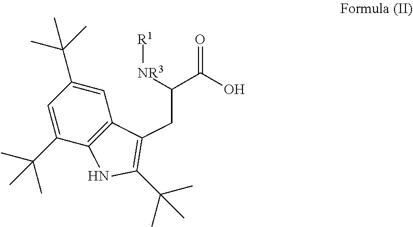
wherein R¹ is a protecting group, a peptide or an amino acid and R³ is H or an alkylsilyl group;
and wherein Formula (III) has the structure: 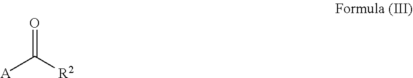
wherein R² is tert-butyl, isobutoxy, tert-butoxy, isobutyl, isopropoxy, isopropyl, or ethoxy; and
A is a halogen.

3. The method of claim 1, wherein R³ is H.

4. The method of claim 1, wherein R² is tert-butyl or isobutoxy.

5. The method of claim 1, wherein R² is tert-butyl, isobutyl, or isopropyl.

6. The method of claim 1, wherein R¹ is a protecting group selected from benzyloxycarbonyl (Cbz), tert-butoxycarbonyl (Boc), 4-methoxy-2,3,6-trimethylbenzene sulphonyl (Mtr), 9-fluorenylmethoxy-carbonyl (Fmoc) and 2,2,2-trichloroethoxycarbonyl (Troc); a peptide or an amino acid.

7. The method of claim 1, wherein R¹ is a peptide or an amino acid.

8. (canceled)

9. The method of claim 1, wherein the R¹ amino acid is a cationic amino acid AA₁.

10. The method of claim 9, wherein AA₁ is arginine.

11. The method of claim 2, further comprising preparing a compound of Formula (II).

12. The method of claim 1, wherein the second moiety comprises one or more protecting groups and/or a C-terminal capping group.

13. The method of claim 12, wherein the C-terminal capping group is of formula —X—Y—Z, wherein:
X is a N atom, which may be substituted by a branched or unbranched C₁-C₁₀ alkyl or aryl group, and this alkyl or aryl group may incorporate up to 2 heteroatoms selected from N, O and S;
Y represents a group selected
from —Rₐ—R_{b}—, —Rₐ—R_{b}—R_{b}— and —R_{b}—R_{b}—Rₐ— wherein
Rₐ is C, O, S or N, and
R_{b} is C; each of Rₐ and R_{b} may be substituted by C₁-C₄ alkyl groups or unsubstituted; and
Z is a group comprising 1 to 3 cyclic groups each of 5 or 6 non-hydrogen atoms, 2 or more of the cyclic groups may be fused and one or more of the cyclic groups may be substituted; the Z moiety incorporates a maximum of 15 non-hydrogen atoms; and wherein
the bond between Y and Z is a covalent bond between Rₐ or R_{b} of Y and a non-hydrogen atom of one of the cyclic groups of Z

14. The method of claim 1, wherein the second moiety is an amino acid comprising AA₃, or a peptide comprising AA₃ as the N-terminal amino acid, wherein AA₃ is a cationic amino acid.

15. The method of claim 1, wherein the second moiety is a compound of Formula (IV)
AA₃-X—Y—Z (IV)
wherein:
AA₃ is a cationic amino acid;
X is a N atom, which may be substituted by a branched or unbranched C₁-C₁₀ alkyl or aryl group, and this alkyl or aryl group may incorporate up to 2 heteroatoms selected from N, O and S;
Y represents a group selected
from —Rₐ—R_{b}—, —Rₐ—R_{b}—R_{b}— and —R_{b}—R_{b}—Rₐ— wherein
Rₐ is C, O, S or N, and
R_{b} is C; each of Rₐ and R_{b} may be substituted by C₁-C₄ alkyl groups or unsubstituted; and
Z is a group comprising 1 to 3 cyclic groups each of 5 or 6 non-hydrogen atoms, 2 or more of the cyclic groups may be fused and one or more of the cyclic groups may be substituted; the Z moiety incorporates a maximum of 15 non-hydrogen atoms; and wherein
the bond between Y and Z is a covalent bond between Rₐ or R_{b} of Y and a non-hydrogen atom of one of the cyclic groups of Z.

16. The method of claim 15, wherein AA₃ is lysine and/or arginine.

17. The method of claim 15, wherein X is unsubstituted, wherein Y is —CH₂—CH₂—, and Z is phenyl.

18-21. (canceled)

22. The method of claim 1, wherein the target peptide has the structure:

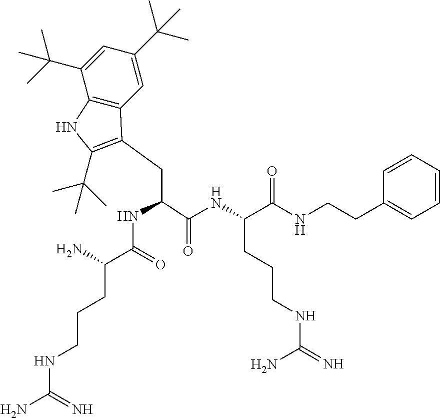 [SMILES: CC(C)(C)c1cc(C(C)(C)C)c2[nH]c(C(C)(C)C)c(C[C@H](NC(=O)[C@@H](N)CCCNC(=N)N)C(=O)N[C@@H](CCCNC(=N)N)C(=O)NCCc3ccccc3)c2c1]

23. A compound of Formula (I) as defined in claim 1.

24. The method of claim 11, wherein R¹ is a peptide or an amino acid and the method comprises coupling said peptide or amino acid to a tri-tert-butyl-tryptophan (Tbt) residue.

25. The method of claim 1, wherein the second moiety is provided in the form of a salt.
